(19) United States
(12) Patent Application Publication
(10) Pub. No.: US 2015/0299614 A1
(43) Pub. Date: Oct. 22, 2015

(54) IMPROVEMENTS IN OR RELATING TO ORGANIC COMPOUNDS

(71) Applicant: Givaudan, S.A., Vernier (CH)

(72) Inventors: Felix Flachsmann, Duebendorf (CH); Andreas Natsch, Uetikon (CH); Jean-Pierre Bachmann, Wädenswil (CH)

(21) Appl. No.: 14/441,063

(22) PCT Filed: Nov. 12, 2013
(86) PCT No.: PCT/EP2013/073560

§ 371 Date: May 6, 2015

(30) Foreign Application Priority Data
Nov. 12, 2012 (GB) 1220341.0

(51) Int. Cl. C11B 9/00; A61Q 5/02; C11D 3/30; A61Q 15/00; C11D 3/50; A61K 8/40; A61Q 5/12

(52) U.S. Cl. CPC C11B 9/0061; A61K 8/40; A61Q 5/02; A61Q 5/12; A61Q 15/00; C11D 3/50; C11D 3/30

(57) ABSTRACT

A fragrance oil comprising a compound of formula (I)

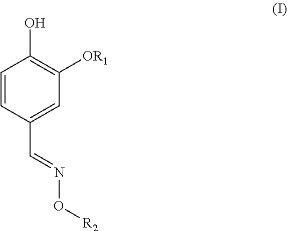

wherein R₁ represents a methyl group or an ethyl group and R₂ represents a methyl or an ethyl group.

The present invention is concerned with fragrance ingredients and with fragrance preparations, for imparting desirable odour notes to consumer products, in particular it is concerned with ingredients and fragrance oils that can be used as replacements in whole or in part for the allergy-disputed compound iso-eugenol.

Iso-eugenol is a widely used ingredient in perfumery. It is valued for its floral and floral-spicy accords such as carnation and lily. In principle, it can be used over a wide range of doses, for example between 0.1 to 10% by weight. However, owing to concerns regarding its potential for eliciting skin allergies, for most practical purposes its use is limited to 200 ppm in consumer products, corresponding to a use of about 0.2 to 2% in a fragrance oil.

Accordingly, there is a need to provide ingredients that can be used in fragrance preparations as a replacement for iso-eugenol or which use in fragrance preparations can substantially reduce the amount of iso-eugenol used in such preparations.

Fragrance ingredients exhibiting floral-spicy notes as iso-eugenol include vanillin and methyl diantilis. However, the use of each of these ingredients is not without its problems. For example, vanillin exhibits a tendency towards discolouration when used in fragrance preparations across a range of consumer bases. Methyl diantilis on the other hand is difficult to use in perfume oils owing to its high water solubility and cannot be formulated easily in sufficiently high amounts for most consumer applications as a result. Besides this, methyl diantilis usage levels above 1 or 2% in fragrance oil has somewhat undesirable hedonic profile, in particular, it begins to impose undesired guaiacol or phenolic notes.

There remains a need for fragrance ingredients that can be used in fragrance oils either as a partial or complete replacement for iso-eugenol, which ingredients are non-allergenic and exhibit good colour stability in consumer product bases upon storage under accelerated conditions.

The invention provides in a first aspect a fragrance oil comprising a compound of formula (I)

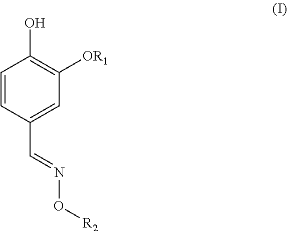

wherein R₁ represents a methyl group or an ethyl group and R₂ represents a methyl or an ethyl group.

Particular compounds of the formula (I) are those wherein R₁ is methyl and R₂ is methyl; or wherein R₁ is methyl and R₂ is ethyl. The most preferred is the compound in which R₁ is ethyl and R₂ represents a methyl group.

The skilled person will appreciate that compounds of formula (I) can be in the form of E and Z isomers about the carbon-nitrogen double bond. When reference is made herein to a compound of formula (I) it is intended to include the E or Z isomer in pure form as well as mixtures thereof.

Compounds of the formula (I) are known in the art. Indeed, certain of the compounds and their odour descriptions are disclosed in EP 0 672 746 B1. In particular, 3-ethoxy-4-hydroxy benzaldehyde o-methyl oxime is described as having a vanilla, spicy odour. However, the applicant was unable to find any reference in the literature relating to perfume compositions containing these specific compounds and consequently applicant was unable to find any reference to the performance of these compounds in perfume compositions or consumer product containing same.

In fact, oxime ethers tend to have much higher odour preception thresholds than their corresponding aldehydes. For example whereas (2E,6Z)-nona-2,6-dienal has a perception threshold of 0.06 ng/l air, its corresponding oxime ether - (1E,2E,6Z)-nona-2,6-dienal O-methyl oxime has a perception threshold of 2.94 ng/l. Similarly, whereas 3-(3-isopropylphenyl)butanal has a perception threshold of 0.07 ng/l, its corresponding oxime ether -E)-3-(3-isopropylphenyl)butanal O-methyl oxime—has a perception threshold of 22.4 ng/l.

Given that oxime ethers are generally much weaker than their corresponding fragrant aldehydes, it is perhaps unsurprising that they are not interesting to perfumers and there is no mention of perfume compositions containing them in the literature. The oxime ethers of the present invention are unusual in that they are uncharacteristically powerful (indeed, in the case of 3-ethoxy-4-hydroxybenzaldehyde O-methyl oxime it is comparable in strength as the powerful odourant ethyl vanillin).

Furthermore, applicant surprisingly found that whereas iso-eugenol as well as certain iso-eugenol “replacers” such as vanillin exhibit colour instability in consumer bases, the oxime ethers of formula (I) are colour stable. By the term “colour stable” is meant that when dosed in a colourless consumer base at 0.02% and held under storage for 4 weeks at 45 ° C., bases containing the compounds of formula (I) remain colourless upon visual inspection. On the other hand, bases containing iso-eugenol and iso-eugenol “replacers” such as vanillin at the same concentration were discoloured, variously appearing to have yellowish, orange, peachy or brown hues.

In a particular embodiment of the present invention there is provided a fragrance oil comprising a compound of formula (I), more particularly the compound wherein R₁ represents ethyl and R₂ represent methyl group, and no iso-eugenol.

In another particular embodiment of the present invention there is provided a fragrance oil, comprising a compound of formula (I) and iso-eugenol, more particularly the compound of formula (I) wherein R₁ represents ethyl and R₂ represent methyl group, wherein the level of iso-eugenol is less than 1% by weight.

In another aspect of the present invention there is provided a consumer product comprising a fragrance oil comprising a compound of formula (I), more particularly the compound wherein R₁ represents ethyl and R₂ represent methyl group, and no iso-eugenol.

In yet another aspect of the present invention there is provided a consumer product, comprising a compound of formula (I) and iso-eugenol, more particularly the compound of formula (I) wherein R₁ represents ethyl and R₂ represent methyl group, wherein the level of iso-eugenol is less than 100 ppm.

Compounds of the formula (I) due to their desirable odour profiles and/or their good colour stability and/or their low allergenic potential provide the perfumer the latitude to use them over wide-ranging amounts, and certainly with less restriction on their usage level than currently imposed on iso-eugenol or indeed iso-eugenol “replacers” such as vanillin or methyl diantilis.

In a particular embodiment of the present invention, the compounds of formula (I) may be employed at levels ranging from 0.1% to20% by weight in a fragrance oil, corresponding to usage levels of 10 to 2000 ppm in a consumer product.

The compounds of formula (I) therefore, have much wider usage levels than iso-eugenol, and can be used in much higher levels than iso-eugenol in fragrance oils and across a wide range of consumer product categories.

In a particular embodiment of the present invention the compounds of formula (I) may be employed in a deodorant or antiperspirant formulation in an amount of 50 to 1000 ppm.

In a particular embodiment of the present invention the compounds of formula (I) may be employed in a rinse-off formulation such as shampoo and hair conditioner in an amount of 100 to 1000 ppm.

In a particular embodiment of the present invention the compounds of formula (I) may be employed in a fabric conditioner formulation in an amount of 50 to 1000 ppm.

In a particular embodiment of the present invention the compounds of formula (I) may be employed in a liquid detergent formulation in an amount of 10 to 1000 ppm.

In a particular embodiment of the present invention the compounds of formula (I) may be employed in a WC rim cleaner formulation in an amount of 10 to 1000 ppm.

In a particular embodiment of the present invention the compounds of formula (I) may be employed in an all purpose cleaner formulation in an amount of 10 to 1000 ppm.

The compounds of formula (I) were also surprisingly found to exhibit a very natural Ylang Ylang-type odour. The use of Ylang Ylang (cananga odorata) extracts is limited to 0.06% by weight in IFRA category 2 products (anti-perspirant/deodorant compositions).

20 In such products in which an Ylang Ylang-type note is desired, it follows that compounds of formula (I) may be used as partial or complete replacers for Ylang Ylang extracts.

Accordingly, the invention provides in another of its aspects a fragrance oil comprising a compound of formula (I) and Ylang Ylang extract, more particularly the compound of formula (I) wherein R₁ represents ethyl and R₂ represent methyl group, wherein the level of Ylang Ylang 25 extract is less than 1% by weight.

In another aspect of the invention there is provided a fragrance oil comprising a compound of formula (I), more particularly the compound wherein R₁ represents ethyl and R₂ represent methyl group, and no Ylang Ylang extract.

In another aspect of the present invention there is provided an antiperspirant or deodorant composition comprising a fragrance oil comprising a compound of formula (I) and Ylang Ylang extract at a level of 600 ppm or less, more particularly the compound of formula (I) wherein R₁ represents ethyl and R₂ represent methyl group.

In another aspect of the present invention there is provided an antiperspirant or deodorant composition comprising a fragrance oil comprising a compound of formula (I), more particularly the compound wherein R₁ represents ethyl and R₂ represent methyl group, and no Ylang Ylang extract.

Applicant is not aware, however, of any single fragrance ingredient that has an odour profile that corresponds precisely to that of iso-eugenol or ylang ylang. The compounds of formula (I) therefore possess an odour profile that is similar to, but not exactly the same as that of iso-eugenol or ylang ylang. As such, perfumers may wish to mix the compounds of formula (I) with certain other perfume ingredients to provide fragrance oils having closer conformance to the odour profile of iso-eugenol or ylang ylang, or even to achieve substantial equivalence to the odour profile of iso-eugenol or ylang ylang. By “substantial equivalence” is meant that a panel of non-experts, e.g. consumers would be unable to detect a difference between a consumer product containing iso-eugenol or ylang ylang and the same product containing a fragrance oil containing a compound of formula (I) of the present invention.

In a particular embodiment of the present invention a compound of formula (I) may form a fragrance oil in combination with one or more of the perfume ingredients selected from the group consisting of Agrumex, Allyl Amyl Glycolate, Ambrettolide, Ambrofix, Amyl Salicylate, Anisic Aldehyde, Benzyl Acetate, Benzyl Benzoate, Benzyl Salicylate, Bornyl Acetate, 4-t-Butyl Cyclohexyl Acetate, Cetalox, Citral, Citronellol, Citronellyl Acetate, Coumarin, Cyclamen Aldehyde, Alpha Damascone, Delta Damascone, Gamma Decalactone, Decanal, Dihydro Myrcenol, Diphenyl Oxide, Dodecanal, Ebanol, Ethyl Linalool, Ethyl Maltol, Ethyl 2-Methylbutyrate, Ethyl Vanillin, Ethylene Brassylate, Eugenol, Evernyl, Fixolide, Florocyclene, Galaxolide, Gardenol, Geraniol, Geranyl Acetate, Habanolide, Hedion, Heliotropine, Z-3-Hexenol, Z-3-Hexenyl Acetate, Z-3-Hexenyl Salicylate, Hexyl Acetate, Alpha Hexyl Cinnamic Aldehyde, Hexyl Salicylate, Indole, Alpha lonone, Beta lonone, Iso E Super, Isoraldeine, Isoeugenol, Jasmacyclene, Javanol, Lilial, Linalool, Linalyl Acetate, Longifolene, Maltol, Manzanate, 2-(1-Mercapto-1-methylethyl)-5-methyl Cyclohexanone, Methyl Benzoate, Methyl Diantilis, Methyl Napthyl Ketone, 2-Methyl Undecanal, Muscenone, Nectaryl, Peach Pure, Peonile, Petalia, Phenoxy Ethyl Isobutyrate, 2-Phenylethyl Acetate, Phenyl Acetaldehyde, 2-Phenyl Ethanol, Radjanol, Rosacetol, Spirogalbanone, Terpineol, Terpinolene, Terpinyl Acetate, Tetrahydro Linalool, 4,7,7-Trimethyl-6-thiabicyclo[3.2.1.]octane, Tricyclal, Tropional, 9-Undecenal, Undecavertol and Vanillin.

In a particular embodiment of the present invention, if a perfume is desired that may be useful to reproduce the character if ylang ylang a compound of the formula (I) may be combined with one or more ingredients selected from the group consisting of Amyl Salicylate, Benzyl Acetate, Benzyl Benzoate, Benzyl Salicylate, Citral, Citronellol, Citronellyl Acetate, para Cresyl Methyl Ether, Alpha Damascone, Delta Damascone, Decanal, Dodecanal, Ethyl Linalool, Ethyl 2-Methylbutyrate, Ethyl Vanillin, Eugenol, Geraniol, Geranyl Acetate, Habanolide, Hedione, Z-3-Hexenol, Z-3-Hexenyl Acetate, Z-3-Hexenyl Salicylate, Hexyl Acetate, Alpha Hexyl Cinnamic Aldehyde, Hexyl Salicylate, Indole, Alpha lonone, Beta lonone, Isoeugenol, Linalool, Linalyl Acetate, Manzanate, Methyl Benzoate, Methyl Diantilis, -2-Phenylethyl Acetate, Phenyl Acetaldehyde, 2-Phenyl Ethanol, Terpineol, Terpinolene, Terpinyl Acetate, Tetrahydro Linalool, Tricyclal and Vanillin.

Fragrance oils need not be limited to a mixture of those ingredients mentioned above, and other ingredients commonly used in perfumery may be employed, for example any of those ingredients described in “Perfume and Flavour Chemicals”, S. Arctander, Allured Publishing Corporation, 1994, Ill., USA, which is incorporated herein by reference.

Fragrance oils of the present invention and consumer products containing same may also contain commonly employed adjuvants. The term “adjuvants” refers to ingredients that may affect the performance of a composition, other than its hedonic performance. For example, an adjuvant may be an ingredient that acts as an aid to processing a fragrance oil or consumer product containing said fragrance oil, or it may improve handling or storage of a fragrance oil or consumer product. It might also be an ingredient that provides additional benefits such as imparting colour or texture. It might also be an ingredient that imparts light resistance or chemical stability to one or more ingredients contained in a fragrance oil or consumer product. A detailed description of the nature and type of adjuvants commonly used in fragrance oils or consumer products cannot be exhaustive, but it has to be mentioned that said ingredients are well known to a person skilled in the art. Examples of adjuvants include solvents and co-solvents; surfactants and emulsifiers; viscosity and rheology modifiers; thickening and gelling agents; preservative materials; pigments, dyestuffs and colouring matters; extenders, fillers and reinforcing agents; stabilisers against the detrimental effects of heat and light, bulking agents, acidulants, buffering agents and antioxidants.

If the fragrance oils employ a mixture of ingredients to reproduce the character of iso-eugenol or ylang ylang, these ingredients will all have different physical or chemical properties, such as volatility and substantivity. Accordingly, for the fragrance oils to retain a coherent character of iso-eugenol or ylang ylang throughout its use cycle, it may be desirable to accelerate or retard the release of one or more ingredients depending on the particular end use. For example, when the fragrance oils are to be used in a laundry detergent composition, it might be desirable to formulate one or more non-substantive ingredients in a delivery vehicle that provides the ingredient(s) with the requisite substantivity such that the character of iso-eugenol or ylang ylang is evident through the whole use cycle from bottle to wash cycle to dry down.

Alternatively, in an end use application where a perfume is expected to bloom upon dilution, such as in the case of a hard-surface cleaner, it will be desirable to formulate the ingredients in such a manner that the individual ingredients develop at substantially the same rate during the cleaning period.

Accordingly, one or more of the ingredients employed in said fragrance oils or consumer products may be formulated in a delivery vehicle to provide a desired effect. Delivery vehicles may include capsule technologies. Alternatively, the delivery vehicle may be in the form of a solid support, e.g. a polymeric support material onto which one or more perfume ingredients may be chemically or physically bound. Still further, one or more perfume ingredients may be dissolved or dispersed in a matrix material, which serves to control the rate at which said ingredient or ingredients emanates. In yet an alternative embodiment, one or more ingredients may be supported on a porous substrate, such as a cyclodextrin or a zeolite or other inorganic material. In a still further embodiment, one or more ingredients may be provided in the form of a pro-perfume, which will react in a suitable environment to release the perfume ingredient in a controlled manner.

Compounds of formula (I) and fragrance oils containing them may be employed in all manner of personal and home care consumer products set out in the IFRA categories. A non-limiting list of applications include a textile treatment product, an ironing aid, a cleaning cloth, a laundry detergent, a cleaning product, in particular, for hard and/or soft surfaces, a household cleaner, a care product, a wash care product, a laundry care product, a room fragrancer, and air freshener, a conditioner, a colorant, a fabric conditioner, a conditioning substrate, a pharmaceutical, a crop protection product, a polish, a food, a cosmetic product, a fertilizer, a building material, an adhesive, a bleach, a decalcifier, an autocare product, floorcare product, cookercare product, leather care product or furniture care product, a scourer, a disinfectant, a fragrancer, a mould remover, fine fragrance, body lotion, skin care preparations, candles, air fresheners, plug ins and toilet soaps.

Particular examples of cleaning products include the toilet cleaners or lavatory cleaners, these products being supplied in the form of powders, blocks, tablets or liquids, or gels, pipe-cleaning products or drain cleaners, universal or all-purpose or general-purpose cleaners, such as those used universally for all hard surfaces in the household and in commerce that can be wiped down wet or damp, sanitary cleaners, oven cleaners or grill cleaners which may be presented in the form of gels or foam sprays, metal polishes, including those supplied as polishing cloths, dipping baths, pastes, and liquids; glass cleaners and window cleaners; all special-purpose cleaning products, for example those for glass-ceramic hobs; carpet cleaners and stain removers.

Particular examples of auto care products include paint preservers, paint polishes, paint cleaners, wash preservers, shampoos for auto washing, auto-wash and wax products, polishes for trim metals, protective films for trim metals, plastics cleaners, tar removers, screen cleaners, engine cleaners and the like.

Particular examples of cosmetic products include cosmetic skincare products, e.g. bath products, skin washing and cleansing products, skincare products, eye makeup, lip care products, nail care products, intimate care products, foot care products; cosmetic products with specific effects, such as sunscreens, tanning products, de-pigmenting products, deodorants, antiperspirants, hair removers, shaving products; cosmetic dental-care products, such as dental and oral care products, toothcare products, cleaners for dental prostheses, adhesives for dental prostheses; cosmetic hair care products, e.g. hair shampoos, hair care products, hair setting products, hair-shaping products, and hair colouring products.

Particular examples of textile treatment products include detergents or fabric conditioners.

Particular examples of air fresheners and room fragrancers include fragrancers for spaces such as autos, cupboards, dishwashers, refrigerators or shoes, and vacuum cleaners.

There now follows a series of examples that serve to illustrate the invention.

### EXAMPLE 1

### Example: Compounds Tested in the KeratinoSens™ Assay for Skin Sensitization

The KeratinoSens™ cell line contains a stable insertion of a Luciferase gene under the control of the ARE-element of the gene AKR1C2. The optimization of this cell line and the standard method to test chemicals has been described in detail in Emter et. al. 2010 (Emter R., Ellis G., Natsch A., 2010 “Performance of a novel keratinocyte-based reporter cell line to screen skin sensitizers in vitro. Toxicol Appl Pharmacol 245, 281-290). Cells were grown for 24 h in 96-well plates. The medium was then replaced with medium containing the test chemical and the solvent Dimethylsulfoxide (DMSO) at a final level of 1%. Each compound was tested at 12 binary dilutions in the range from 0.98 to 2000 μM. In each repetition, three parallel replicate plates were run with this same set-up and a fourth parallel plate was prepared for cytotoxicity determination using an MTT assay. Cells were incubated for 48 h with the test agents, and then luciferase activity and cytotoxicity were determined. This full procedure was repeated at least three times for each chemical. For each chemical in each repetition and at each concentration, the gene induction compared to DMSO controls and the wells with statistically significant induction over the threshold of 1.5 (i.e. 50% enhanced gene activity) were determined. Furthermore the maximal fold-induction (Iₘₐₓ) and the EC1.5 value (concentration in μM for induction above the threshold) were calculated. Chemicals are rated as positive (i.e. likely skin-sensitizers) in the assay if the following three criteria are fulfilled (i) The EC1.5 value is below 1000 μM in all three repetitions or in at least 2 repetitions, (ii) at the lowest concentration with a gene induction above 1.5 fold (i.e. at the EC 1.5 determining value), the cellular viability is above 70% and (iii) there is an apparent overall dose-response for luciferase induction, which is similar between the repetitions.

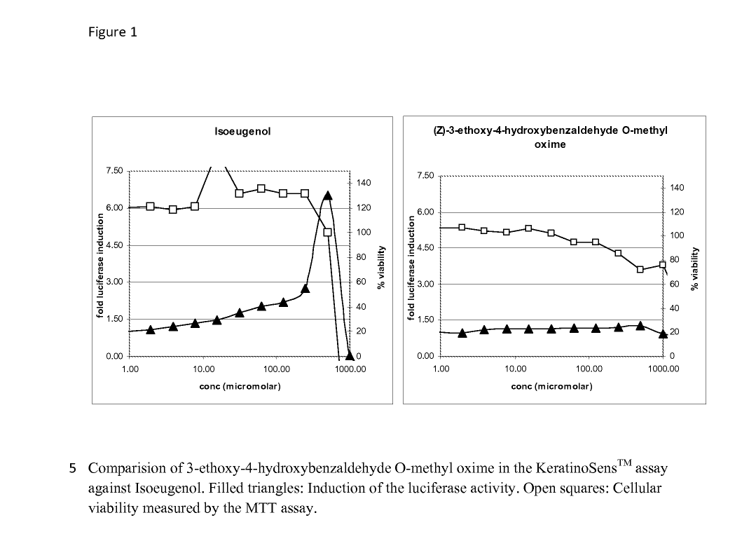

FIG. 1 shows the gene-induction and cell viability curves for Isoeugenol and ethoxy-4-hydroxybenzaldehyde O-methyl oxime (compound of formula (I) wherein R₁ is ethyl and R₂ is methyl). The latter did not induce luciferase activity above the 1.5-fold threshold and is thus rated as non-sensitizing by this assay. It also has no significant cytotoxicity up to 1000 micromolar. Isoeugenol on the other hand clearly induces the gene already in the lower micromolar range, indicating it is a sensitizing compound. In addition at concentrations above 500 micromolar it is cytotoxic. These results show that 3-ethoxy-4-hydroxybenzaldehyde O-methyl oxime can be used in perfume formulations instead of Isoeugenol to reduce the sensitization risk to the consumer.

### Example 2: Colour Stability in Consumer Products

### Example 2a: Colour Stability in Fine Fragrance

A solution of the test compound (compound of formula (I) specified in the Table below) was prepared in perfumery grade ethanol (96% EtOH, 4% water) at 1 and 0.5% wt/wt. The solutions were placed in 2.5 ml transparent glass vials closed with plastic lids. The vials were placed behind a window exposed to indoor daylight. The colour of the solutions was inspected visually over a 1 month period.

| | Colour of ethanol solution | |
|---|---|---|
| Test compound | after 1 month | Odour |
| Vanillin | yellow | vanilla |
| Ethyl Vanillin | yellow | vanilla |
| Cpd I (R₁ = Et, R₂ = Me) | colourless | Spicy vanilla floral |

The ethanol solutions perfumed with compound I of the present invention would have passed the requirements for colourless fine fragrance at both concentrations after 1 month, whereas the ones containing vanillin or ethyl vanillin had developed an unacceptable yellow colour.

### Example 2b: Colour Stability in Bar Soap

A standard tallow toilet soap sample was prepared by adding 2% wt/wt of a 10% wt/wt solution in dipropylene glycol (DPG) of either ethyl vanillin or compound I (R₁=Et, R₁=Me) to the soap base. The extruded 24×8 mm soap string was cut into 8 cm pieces, which were placed in a thermostated oven for 1 month at 45° C. A control sample containing no fragrance was added to the stability test series. After one month, the colour of the ethyl vanillin containing soap had turned into a chocolate brown, the odour was vanilla like. The colour of the soap containing compound I remained as control, its odour was vanilla spicy floral like.

1. A fragrance oil comprising a compound of formula (I) 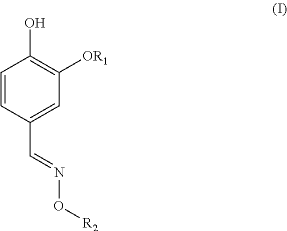
wherein R₁ represents a methyl group or an ethyl group and R₂ represents a methyl or an ethyl group.

2. The fragrance oil according to claim 1 comprising the compound of formula (I) wherein R₁ is ethyl and R₂ represents a methyl group.

3. The fragrance oil according to claim 1 that contains from less than 1% by weight iso-eugenol.

4. The fragrance oil according to claim 1 wherein the compound of the formula (I) is employed at a level ranging between 0.1% to 20%.

5. The fragrance oil according to claim 1 additionally comprising one or more perfume ingredients selected from the group consisting of Agrumex, Allyl Amyl Glycolate, Ambrofix, Amyl Salicylate, Anisic Aldehyde, Benzyl Acetate, Benzyl Benzoate, Benzyl Salicylate, Bornyl Acetate, 4-t-Butyl Cyclohexyl Acetate, Cetalox, Citral, Citronellol, Citronellyl Acetate, Coumarin, Cyclamen Aldehyde, Alpha Damascone, g-Decalactone, Decanal, Dihydro Myrcenol, Diphenyl Oxide, Dodecanal, Ethyl Linalool, Ethyl 2-Methylbutyrate, Ethyl Vanillin, Ethylene Brassylate, Eugenol, Evernyl, Fixolide, Florocyclene, Galaxolide, Gardenol, Geraniol, Geranyl Acetate, Habanolide, Hedion, Heliotropine, Z-3-Hexenol, Z-3-Hexenyl Acetate, Hexyl Acetate, a-Hexyl Cinnamic Aldehyde, Hexyl Salicylate, Indole, b-Ionone, Iso E Super, Isoraldeine 70, Isoeugenol, Jasmacyclene, Lilial, Linalool, Linalyl Acetate, Longifolene, Methyl Diantilis, 2-Methyl Undecanal, Muscenone, Peach Pure, Peonile, Phenoxy Ethyl Isobutyrate, 2-Phenylethyl Acetate, 2-Phenyl Ethanol, Rosacetol, Terpineol, Terpinolene, Terpinyl Acetate, Tetrahydro Linalool, Tricyclal, Tropional, Undecavertol and Vanillin.

6. The fragrance oil according to claim 1 comprising Ylang Ylang extract, wherein the level of Ylang Ylang extract is less than 1% by weight.

7. A consumer product comprising a fragrance oil comprising the compound of formula (I) as defined in claim 1.

8. The consumer product according to claim 7 in the form of a deodorant or antiperspirant formulation wherein the compound of formula (I) is present in an amount of 50 to 1000 ppm.

9. The consumer product according to claim 7 in the form of a rinse-off formulation, optionally shampoo or hair conditioner, wherein the compound of formula (I) is present in an amount of 100 to 1000 ppm.

10. The consumer product according to claim 7 in the form of a fabric care formulation, optionally fabric conditioner or tumble dryer sheets, wherein the compound of formula (I) is present in an amount of 100 to 1000 ppm.

11. The consumer product according to claim 7 in the form of a liquid detergent formulation wherein the compound of formula (I) is present in an amount of 10 to 1000 ppm.

12. The consumer product according to claim 7 in the form of a WC rim cleaner formulation wherein the compound of formula (I) is present in an amount of 10 to 1000 ppm.

13. The consumer product according to claim 7 in the form of an all purpose cleaner formulation wherein the compound of formula (I) is present in an amount of 10 to 1000 ppm.

14. The consumer product according to claim 7 wherein R₁ is ethyl and R₂ represents a methyl group.
